(19) United States
(12) Patent Application Publication
(10) Pub. No.: US 2025/0009766 A1
(43) Pub. Date: Jan. 9, 2025

(54) CANNABIGEROL FOR TREATMENT OF SEIZURES AND EPILEPSY

(71) Applicant: The Regents of the University of California, Oakland, CA (US)

(72) Inventors: Michael ROGAWSKI, Oakland, CA (US); Ashish DHIR, Oakland, CA (US); Mark MASCAL, Oakland, CA (US)

(21) Appl. No.: 18/706,266

(22) PCT Filed: Nov. 1, 2022
(86) PCT No.: PCT/US2022/079033

§ 371 Date: Apr. 30, 2024

Related U.S. Application Data
(60) Provisional application No. 63/274,329, filed on Nov. 1, 2021.

(51) Int. Cl. A61K 31/00; A61P 25/08

(52) U.S. Cl. CPC A61K 31/658; A61P 25/08

(57) ABSTRACT

The present invention provides a method of treating or mitigating seizures or epilepsy comprising compounds as described herein.

### CROSS-REFERENCES TO RELATED APPLICATIONS

This application claims priority to U.S. Provisional Application No. 63/274,329, filed Nov. 1, 2021, which is incorporated herein in its entirety for all purposes.

### BACKGROUND

Cannabidiol (CBD), a biologically active constituent of the cannabis (hemp) plant, has been approved for the treatment of seizures associated with Lennox-Gastaut syndrome, Dravet syndrome, and tuberous sclerosis complex. CBD protects against tonic hind limb extension (THLE) in the mouse maximal electroshock (MES) test, a standard model used in the identification of antiseizure agents. Although CBD has been extensively studied, many other plant cannabinoids have not been assessed for potential utility in epilepsy therapy. The current study determined the effectiveness and potencies of cannabigerol (CBG) and cannabichromene (CBC) in the mouse MES test.

### BRIEF SUMMARY OF THE INVENTION

In one embodiment, the present invention provides a method of treating or mitigating a seizure or epilepsy, comprising administering to a subject in need thereof, a therapeutically effective amount of a compound of Formula I:

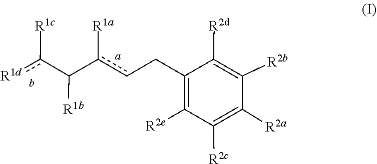

or a pharmaceutically acceptable salt thereof, thereby treating the seizure or epilepsy, wherein:
R^{1a} and R^{1d} are each independently —CO₂R^{1e}, C₁₋₂₀ alkyl, C₂₋₂₀ alkenyl or C₂₋₂₀ alkynyl, wherein at least one of R^{1a} or R^{1d} is methyl or isopropyl;
R^{1b} and R^{1c} are each independently hydrogen or oxygen;
alternatively, when R^{1b} is oxygen, R^{1b} is combined with R^{1a} and the atoms to which they are attached to form an epoxide ring;
alternatively, R^{1b} is combined with R^{1d} and the atoms to which they are attached to form a C₄₋₈ cycloalkyl, wherein the cycloalkyl is substituted with 1-3 R^{1e} groups;
R^{1e} is H, C₁₋₂₀ alkyl, C₂₋₂₀ alkenyl or C₂₋₂₀ alkynyl;
R^{2a} is —OR^{2f}, C₁₋₂₀ alkyl, C₂₋₂₀ alkenyl or C₂₋₂₀ alkynyl;
R^{2b} and R^{2c} are each independently hydrogen, halogen, —OR^{2f}, —NR^{2f}R^{2g}, or —C(O)OR^{2f};
R^{2a} and R^{2e} are each independently —OH, —OC(O)R^{2f}, —OR^{2f}, C₁₋₂₀ alkyl, C₂₋₂₀ alkenyl or C₂₋₂₀ alkynyl;
R^{2f} and R^{2g} are each independently hydrogen, C₁₋₂₀ alkyl, C₂₋₂₀ alkenyl or C₂₋₂₀ alkynyl; and
dashed lines a and b are each independently absent or a bond.

In another embodiment, the present invention provides a method of reducing the frequency of seizures, comprising administering to a subject in need thereof, a therapeutically effective amount of a compound of the present invention, or a pharmaceutically acceptable salt thereof, without inducing hypnotic effects in the subject, thereby reducing the frequency of seizures.

### BRIEF DESCRIPTION OF THE DRAWINGS

FIG. 1 shows response curve of pure crystalline chemically synthesized cannabigerol (CBG) in mouse maximal electroshock test. Male CF1 mice were administered with a single intraperitoneal injection of 100 mg/kg CBG. Following 15, 30, 60, 120, and 240 min after its administration, these animals were challenged with maximal electroshock (MES). Different groups of animals were used for different time points. CBG started to show protection from tonic hindlimb extension (THLE) caused by maximal electroshock during 15 and 30 min and the maximum response could be seen between 60-120 min. The response slightly declined at 240 min after its administration. Each group represents eight mice.

FIG. 2 shows dose-response relationships for the protective effect of cannabigerol (CBG) and cannabidiol (CBD) in the MES test using CF1 mice. CBG or CBD was obtained from decarboxylation of their acidic precursor CBGA or CBDA, respectively, and was administered 60 min before electrical stimulation. Data points indicate the percentage of animals protected from THLE. Each point represents eight mice. Both CBG and CBD protected animals from Tonic Hindlimb Extension (THLE) in the MES test.

FIG. 3 shows response curve of cannabigerol (CBG) in mouse maximal electroshock test compared to cannabidiol (CBD).

FIG. 4 shows response curve of cannabigerol (CBG) in mouse maximal electroshock test compared to cannabidiol (CBD).

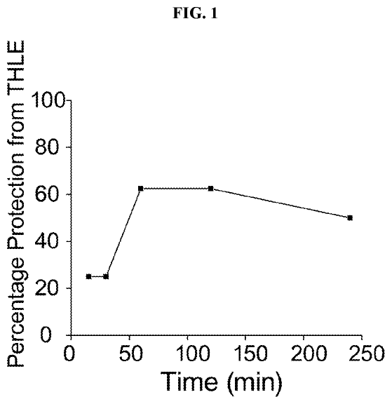

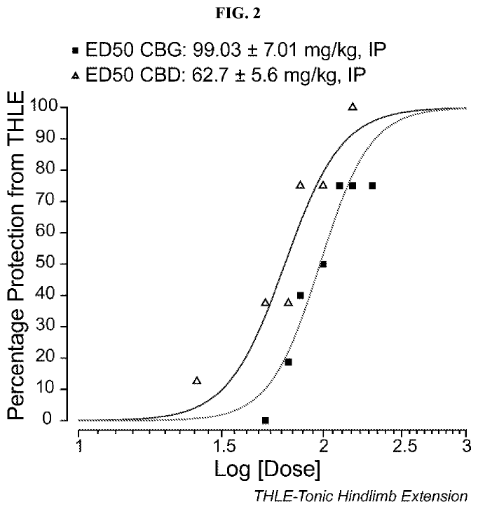

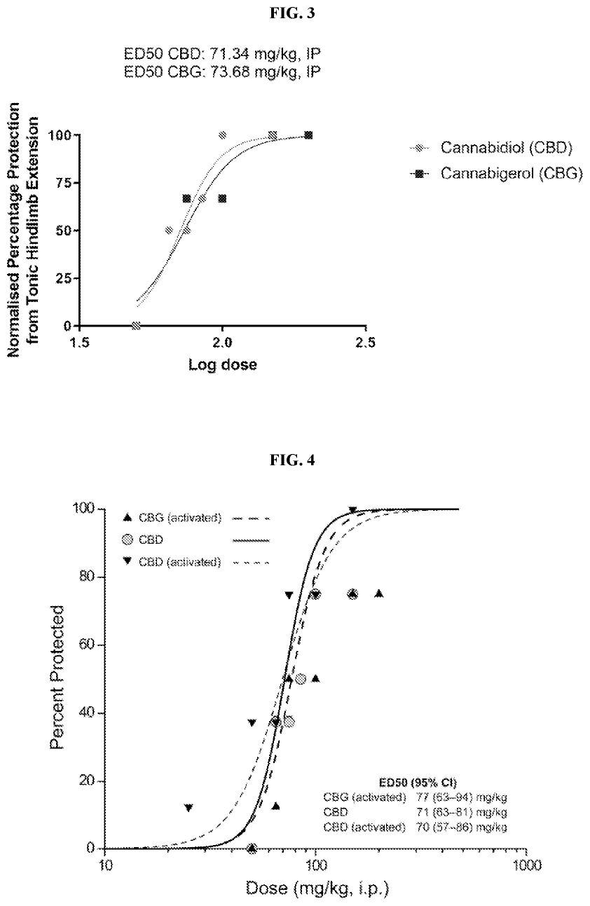

### DETAILED DESCRIPTION

### I. General

The present invention provides a method of treating or mitigating seizure, treating epilepsy, as well as a method of reducing the frequency of seizures, using cannabigerol or 6′,7′-dihydrocannabigerol and analogs thereof.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“A,” “an,” or “the” not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂-6, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Halogen” refers to fluorine, chlorine, bromine and iodine.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃-6, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2]bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

“Epoxide” refers to a three-atom cyclic ether with the following structure:

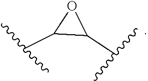 [SMILES: *C1OC1*]

“Isomers” refers to compounds with same chemical formula but different relative orientation between the atoms in the molecule, leading to distinct chemical structures. Isomers include structural isomers and stereoisomers. Examples of structural isomers include, but are not limited to tautomers and regioisomers. Examples of stereoisomers include but are not limited to diastereomers and enantiomers.

“Pharmaceutically acceptable salt” refers to a compound in salt form, wherein the compound are suitable for administration to a subject. Representative pharmaceutically acceptable salts include salts of acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic and xinafoic acid, and the like.

“Subject” refers to animals such as mammals, including, but not limited to, primates (e.g., humans), cows, sheep, goats, horses, dogs, cats, rabbits, rats, mice and the like. In certain embodiments, the subject is a human.

“Mitigating” refers to a reduction in the severity of, or the weakening of a condition or symptom.

“Treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter; including, e.g., the result of a physical examination.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Subject” refers to animals such as mammals, including, but not limited to, primates (e.g., humans), cows, sheep, goats, horses, dogs, cats, rabbits, rats, mice and the like. In certain embodiments, the subject is a human. In some embodiments, the subject is a companion animal.

### III. Method of Treatment

The compounds of the present invention can be used for treating or mitigating a seizure and treating epilepsy. The compounds of the present invention can also be used for reducing the frequency of seizures.

In some embodiments, the compounds of the present invention are used for treating or mitigating convulsant effects. In some embodiments, the compounds of the present invention are used for treating or mitigating seizures. In some embodiments, the compounds of the present invention are used for treating or mitigating epilepsy. In some embodiments, the compounds of the present invention have anti-convulsant properties.

### A. Method of Treating or Mitigating Seizure

In some embodiments, the present invention provides a method of treating or mitigating epilepsy or a seizure, comprising administering to a subject in need thereof, a therapeutically effective amount of a low abuse potential cannabinoid.

In some embodiments, the present invention provides a method of treating or mitigating epilepsy or a seizure, comprising administering to a subject in need thereof, a therapeutically effective amount of a compound of the present invention. In some embodiments, the present invention provides a method of treating or mitigating a seizure, comprising administering to a subject in need thereof, a therapeutically effective amount of a compound of the present invention.

In some embodiments, the present invention provides a method of treating or mitigating a seizure or epilepsy, comprising administering to a subject in need thereof, a therapeutically effective amount of a compound of Formula I:

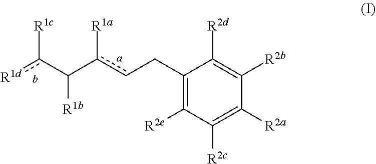

or a pharmaceutically acceptable salt thereof, thereby treating the seizure or epilepsy, wherein:
R^{1a} and R^{1d} are each independently —CO₂R^{1e}, C₁₋₂₀ alkyl, C₂₋₂₀ alkenyl or C₂₋₂₀ alkynyl, wherein at least one of R^{1a} or R^{1d} is methyl or isopropyl;
R^{1b} and R^{1c} are each independently hydrogen or oxygen;
alternatively, when R^{1b} is oxygen, R^{1b} is combined with R^{1a} and the atoms to which they are attached to form an epoxide ring;
alternatively, R^{1b} is combined with R^{1d} and the atoms to which they are attached to form a C₄₋₈ cycloalkyl, wherein the cycloalkyl is substituted with 1-3 R^{1e} groups;
R^{1e} is H, C₁₋₂₀ alkyl, C₂₋₂₀ alkenyl or C₂₋₂₀ alkynyl;
R^{2a} is —OR^{2F}, C₁₋₂₀ alkyl, C₂₋₂₀ alkenyl or C₂₋₂₀ alkynyl;
R^{2b} and R^{2c} are each independently hydrogen, halogen, —OR^{2F}, —NR^{2f}R^{2g}, or —C(O)OR^{2f};
R^{2d} and R^{2e} are each independently —OH, —OC(O)R^{2f}, —OR^{2f}, C₁₋₂₀ alkyl, C₂₋₂₀ alkenyl or C₂₋₂₀ alkynyl;
R^{2f} and R^{2g} are each independently hydrogen, C₁₋₂₀ alkyl, C₂₋₂₀ alkenyl or C₂₋₂₀ alkynyl; and
dashed lines a and b are each independently absent or a bond.

In some embodiments, R^{1a} and R^{1d} are each independently —CO₂R^{1e}, C₁₋₂₀ alkyl, C₂₋₂₀ alkenyl or C₂₋₂₀ alkynyl, wherein at least one of R^{1a} or R^{1d} is methyl or isopropyl; R^{1b} and R^{1c} are each independently hydrogen or oxygen; alternatively, when R^{1b} is oxygen, R^{1b} is combined with R^{1a} and the atoms to which they are attached to form an epoxide ring; alternatively, R^{1b} is combined with R^{1d} and the atoms to which they are attached to form a C₄₋₈ cycloalkyl, wherein the cycloalkyl is substituted with 1-3 R^{1e} groups; R^{1e} is H, C₁₋₂₀ alkyl, C₂₋₂₀ alkenyl or C₂₋₂₀ alkynyl; R^{2a} is —OR^{2F}, C₁₋₂₀ alkyl, C₂₋₂₀ alkenyl or C₂₋₂₀ alkynyl; R^{2b} and R^{2e} are each independently hydrogen, halogen, —OR^{2f}, or —NR^{2f}R^{2g}; R^{2a} and R^{2e} are each independently —OH, —OC(O)R^{2f}, —OR^{2f}, C₁₋₂₀ alkyl, C₂₋₂₀ alkenyl or C₂₋₂₀ alkynyl; R^{2f} and R_{2g} are each independently hydrogen, C₁₋₂₀ alkyl, C₂₋₂₀ alkenyl or C₂₋₂₀ alkynyl; and dashed lines a and b are each independently absent or a bond.

In some embodiments, R^{1a} and R^{1d} are each independently —CO₂R^{1e}, C₁₋₂₀ alkyl, C₂₋₂₀ alkenyl or C₂₋₂₀ alkynyl, wherein at least one of R^{1a} or R^{1d} is methyl or isopropyl; R^{1b} and R^{1c} are each independently hydrogen or oxygen; alternatively, when R^{1b} is oxygen, R^{1b} is combined with R^{1a} and the atoms to which they are attached to form an epoxide ring; alternatively, R^{1b} is combined with R^{1d} and the atoms to which they are attached to form a C₄₋₈ cycloalkyl, wherein the cycloalkyl is substituted with 1-3 R^{1e} groups; and R^{1e} is H, C₁₋₂₀ alkyl, C₂₋₂₀ alkenyl or C₂₋₂₀ alkynyl.

In some embodiments, R^{1a} and R^{1d} are each independently —CO₂R^{1e}, C₁₋₂₀ alkyl, C₂₋₂₀ alkenyl or C₂₋₂₀ alkynyl. In some embodiments, R^{1a} and R^{1d} are each independently C₁₋₂₀ alkyl, C₂₋₂₀ alkenyl or C₂₋₂₀ alkynyl. In some embodiments, R^{1a} and R^{1d} are each independently C₁₋₂₀ alkyl or C₂₋₂₀ alkenyl. In some embodiments, R^{1a} and R^{1d} are each independently C₁₋₂₀ alkyl. In some embodiments, R^{1a} is C₁₋₂₀ alkyl, and R^{1d} is C₂₋₂₀ alkenyl.

In some embodiments, R^{1a} is C₁₋₂₀ alkyl. In some embodiments, R^{1a} is methyl, ethyl, propyl, isopropyl, butyl, iso-butyl, sec-butyl, tert-butyl, or pentyl. In some embodiments, R^{1a} is methyl, ethyl, propyl, or isopropyl. In some embodiments, R^{1a} is methyl.

In some embodiments, R^{1d} is C₁₋₂₀ alkyl or C₂₋₂₀ alkenyl. In some embodiments, R^{1d} is C₁₋₂₀ alkyl. In some embodiments, R^{1d} is sec-butyl. In some embodiments, R^{1d} is C₂₋₂₀ alkenyl. In some embodiments, R^{1d} is 2-methyl-prop-1-enyl or isopropenyl. In some embodiments, R^{1d} is 2-methyl-prop-1-enyl. In some embodiments, R^{1d} is isopropenyl. In some embodiments, R^{1a} is methyl and R^{1d} is isopropenyl. In some embodiments, R^{1d} is isopropenyl. In some embodiments, R^{1a} is methyl and R^{1d} is 2-methyl-prop-1-enyl.

In some embodiments, R^{1b} and R^{1c} are each independently hydrogen or oxygen. In some embodiments, R^{1b} is oxygen, or R^{1b} is combined with R^{1a} and the atoms to which they are attached to form an epoxide ring. In some embodiments, R^{1b} is combined with R^{1d} and the atoms to which they are attached to form a C₄₋₈ cycloalkyl. In some embodiments, R^{1b} is oxygen. In some embodiments, R^{1b} is hydrogen. In some embodiments, R^{1c} is oxygen. In some embodiments, R^{1c} is hydrogen.

In some embodiments, R^{2a} is —OR^{2f}, C₁₋₂₀ alkyl, C₂₋₂₀ alkenyl or C₂₋₂₀ alkynyl; R^{2b} and R^{2c} are each independently hydrogen, halogen, —OR^{2f}, or —NR^{2f}R^{2g}; R^{2a} and R^{2e} are each independently —OH, —OC(O)R^{2f}, —OR^{2f}, C₁₋₂₀ alkyl, C₂₋₂₀ alkenyl or C₂₋₂₀ alkynyl; and R^{2f} and R^{2g} are each independently hydrogen, C₁₋₂₀ alkyl, C₂₋₂₀ alkenyl or C₂₋₂₀ alkynyl.

In some embodiments, R^{2a} is —OR^{2f}, C₁₋₂₀ alkyl, C₂₋₂₀ alkenyl or C₂₋₂₀ alkynyl. In some embodiments, R^{2a} is C₁₋₂₀ alkyl, C₂₋₂₀ alkenyl or C₂₋₂₀ alkynyl. In some embodiments, R^{2a} is C₁₋₂₀ alkyl. In some embodiments, R^{2a} is C₄—is alkyl. In some embodiments, R^{2a} is C₄₋₁₀ alkyl. In some embodiments, R^{2a} is butyl, pentyl, isopentyl, hexyl, 2-methylhex-2-yl, heptyl, 3-methylhept-2-yl, or octyl. In some embodiments, R^{2a} is pentyl, isopentyl, hexyl, 2-methylhex-2-yl, heptyl, or 3-methylhept-2-yl.

In some embodiments, R^{2a} is —OR^{2f}, wherein R^{2f} is hydrogen or C₁₋₂₀ alkyl. In some embodiments, R^{2a} is —OR^{2F}, wherein R^{2f} is C₁₋₂₀ alkyl. In some embodiments, R^{2f} is butyl, pentyl, isopentyl, hexyl, 2-methylhex-2-yl, heptyl, 3-methylhept-2-yl, or octyl. In some embodiments, R^{2f} is pentyl, isopentyl, hexyl, 2-methylhex-2-yl, heptyl, or 3-methylhept-2-yl.

In some embodiments, R^{2b} and R^{2c} are each independently hydrogen, halogen, —OR^{2f}, —NR^{2f}R^{2g}, or —C(O)OR^{2f}. In some embodiments, R^{2b} and R^{2c} are each independently hydrogen, halogen, —OR^{2F}, or —NR^{2f} R^{2g}. In some embodiments, R^{2b} and R^{2c} are each independently hydrogen, halogen, or —OR^{2f}. In some embodiments, R^{2b} and R^{2c} are each independently hydrogen, F, Cl, —OH, or —O—C₁₋₆alkyl. In some embodiments, R^{2b} and R^{2c} are each independently hydrogen or F. In some embodiments, R^{2b} and R^{2c} are each hydrogen. In some embodiments, R^{2b} and R^{2c} are both F.

In some embodiments, R^{2b} is —C(O)OR^{2f}; R^{2c} is hydrogen; and R^{2f} is hydrogen. In some embodiments, R^{2b} is —C(O)OH and R^{2c} is hydrogen. In some embodiments, the compound of Formula I is cannabigerolic acid (CBGA).

In some embodiments, R^{2d} and R^{2e} are each independently —OH, —OC(O)R^{2f}, —OR^{2f} C₁₋₂₀ alkyl, C₂₋₂₀ alkenyl or C₂₋₂₀ alkynyl. In some embodiments, R^{2d} and R^{2e} are each independently —OH, —OC(O)R^{2f}, or —OR^{2f}. In some embodiments, R^{2a} and R^{2e} are each independently —OH, —OC(O)R^{2f}, or —OR^{2f}; and R^{2f} is hydrogen, or C₁₋₂₀ alkyl. In some embodiments, R^{2a} and R^{2e} are each independently —OH, —OC(O)Me, —OC(O)Et, —OMe, —OEt, —OPr, or —OBu. In some embodiments, R^{2a} and R^{2e} are each independently —OH, —OC(O)Me, or —OMe. In some embodiments, R^{2d} and R^{2e} are each —OH.

In some embodiments, R^{2f} and R^{2g} are each independently hydrogen, C₁₋₂₀ alkyl, C₂₋₂₀ alkenyl or C₂₋₂₀ alkynyl. In some embodiments, R^{2f} and R^{2g} are each independently hydrogen, C₁₋₂₀ alkyl, or C₂₋₂₀ alkenyl. In some embodiments, R^{2f} and R^{2g} are each independently hydrogen or C₁₋₂₀ alkyl. In some embodiments, R^{2f} and R^{2g} are each independently hydrogen, methyl, ethyl, propyl, or isopropyl. In some embodiments, R^{2f} and R^{2g} are each independently hydrogen or methyl. In some embodiments, R^{2f} and R^{2g} are both hydrogen. In some embodiments, R^{2f} and R^{2g} are both methyl.

In some embodiments, dashed lines a and b are each independently absent or a bond.

In some embodiments, dashed line a is absent or a bond. In some embodiments, a is absent. In some embodiments, dashed line a is a bond. In some embodiments, dashed line b is absent or a bond. In some embodiments, b is absent. In some embodiments, b is a bond.

In some embodiments, R^{1a} and R^{1d} are each independently C₁₋₂₀ alkyl, wherein at least one of R^{1a} or R^{1d} is methyl or isopropyl; R^{1b} and R^{1c} are each hydrogen; R^{2a} is —OR^{2f} or C₁₋₂₀ alkyl; R^{2b} and R^{2c} are each independently hydrogen, or —C(O)OR^{2f}; R^{2d} and R^{2e} are each independently —OH, —OC(O)R^{2f}, or —OR^{2f}; each R^{2f} and R^{2g} is hydrogen, or C₁₋₂₀ alkyl; and dashed lines a and b are each independently absent or a bond. In some embodiments, R^{1a} and R^{1d} are each independently C₁₋₂₀ alkyl, wherein at least one of R^{1a} or R^{1d} is methyl or isopropyl; R^{1b} and R^{1c} are each hydrogen; R^{2a} is —OR^{2f} or C₁₋₂₀ alkyl; R^{2b} and R^{2c} are each independently hydrogen, or —C(O)OH; R^{2d} and R^{2e} are each —OH; R^{2f} is C₁₋₂₀ alkyl; and dashed lines a and b are each independently absent or a bond.

In some embodiments, the compound is Formula Ia:

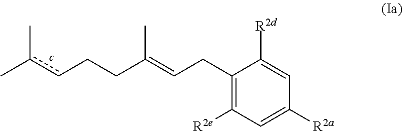

or a pharmaceutically acceptable salt thereof,
wherein
R^{2a} is C₁₋₂₀ alkyl, C₂₋₂₀ alkenyl or C₂₋₂₀ alkynyl;
R^{2d} and R^{2e} are each independently —OH, —OC(O)R^{2f}, or —OR^{2f};
each R^{2f} is independently hydrogen, C₁₋₂₀ alkyl, C₂₋₂₀ alkenyl or C₂₋₂₀ alkynyl; and dashed line c is absent or a bond.

In some embodiments, the compound is Formula Ia:

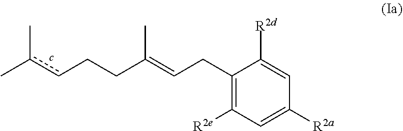

or a pharmaceutically acceptable salt thereof,
wherein
R^{2a} is C₁₋₂₀ alkyl;
R^{2d} and R^{2e} are each —OH; and
dashed line c is absent or a bond.

In some embodiments, the compound is Formula Ib:

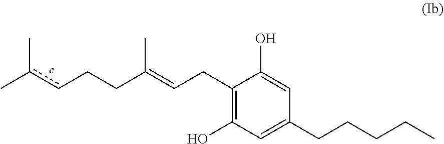

or a pharmaceutically acceptable salt thereof,
wherein dashed line c is absent or a bond.

In some embodiments, the compound is:

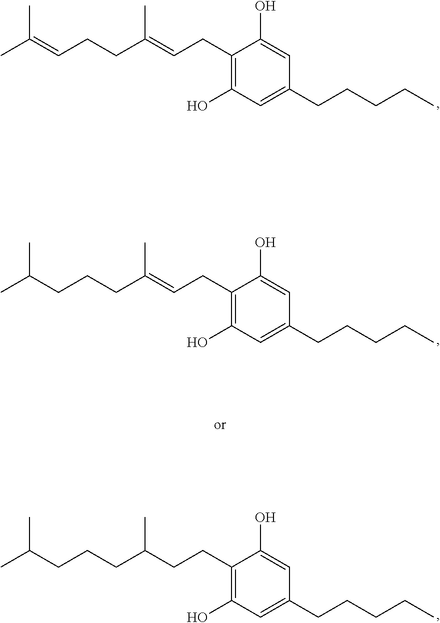

or a pharmaceutically acceptable salt thereof.

In some embodiments, the compound is:

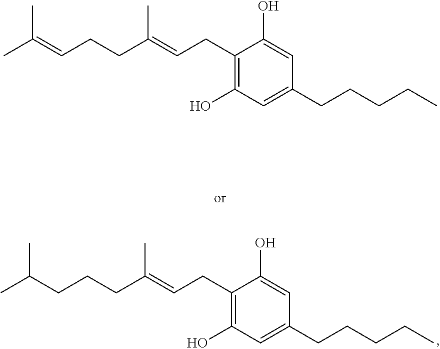

or a pharmaceutically acceptable salt thereof.

In some embodiments, the compound of Formula I is cannabigerol:

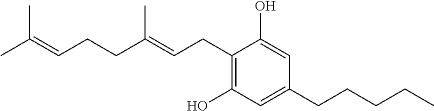 [SMILES: CCCCCc1cc(O)c(C/C=C(\C)CCC=C(C)C)c(O)c1]

or a pharmaceutically acceptable salt thereof.

In some embodiments, the compound of Formula I is cannabigerol:

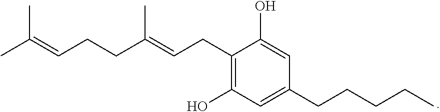 [SMILES: CCCCCc1cc(O)c(C/C=C(\C)CCC=C(C)C)c(O)c1]

In some embodiments, the subject is not being treating for pain, bipolar disorder, or a unipolar mood disorder with the compound of Formula I.

In some embodiments, electroencephalogram (EEG), high-density EEG, computerized tomography (CT) scan, magnetic resonance imaging (MRI), functional MRI (fMRI), positron emission tomography (PET), single-photo emission computerized tomography (SPECT), subtraction ictal SPECT coregistering to MRI (SISCOM), statistical parametric mapping (SPMJ), curry analysis, and magnetoencephalography (MEG) can be used to determine the treating or mitigating of a seizure.

Epileptic syndromes often present with many different types of seizures, any of which may be treated herein. Epileptic seizures are generally categorized as: 1) focal onset seizures, where the seizure originates within networks limited to only one hemisphere, such as aura, motor, autonomic, and awareness/responsiveness seizures (e.g., focal impaired awareness seizure and focal awareness seizure), including ictal atonia seizures; 2) generalized onset seizures, where the seizure arises within and rapidly engages bilaterally distributed networks, such as generalized tonic-clonic seizures, absence seizures, clonic seizures, tonic seizures, atonic seizures, and myoclonic seizures; and 3) unknown onset seizures where the origination site of the seizure cannot be determined. Subjects with epilepsy can also present with “mixed seizures,” which are those defined by the existence of both generalized and focal onset seizures in the same subject.

The epilepsy treated herein may be childhood epilepsy, which refers to the many different syndromes and genetic mutations that can occur to cause epilepsy in childhood. Examples of childhood epilepsy include, but are not limited to, Dravet Syndrome, myoclonic-absence epilepsy, Lennox-Gastaut syndrome, generalized epilepsy of unknown origin, CDKL5 deficiency disease, PCDH19-epilepsy, continuous spikes and waves during sleep (CSWS), electrical status epilepticus during slow-wave sleep (ESES) epilepsy, Aicardi syndrome, Ohtahara syndrome, bilateral polymicrogyria, Dup15q syndrome, SNAP25-associated epilepsy, febrile infection related epilepsy syndrome (FIRES), benign rolandic epilepsy, juvenile myoclonic epilepsy, myoclonic astatic epilepsy (Doose syndrome), infantile spasm (West syndrome), and Landau-Kleffner syndrome.

The epilepsy treated herein may be drug resistant epilepsy (DRE). Examples of types of epilepsy which frequently fall into the drug resistant epilepsy category include, but are not limited to, Dravet syndrome, Lennox-Gastaut syndrome, myoclonic absence seizures, febrile infection related epilepsy syndrome (FIRES), treatment-resistant adult focal epilepsy (TRAFE), and PCDH19-epilepsy.

The epilepsy treated herein may also be those epileptic disorder etiologies that present in the form of atonic seizures, which involve the loss of muscle tone, causing the person to fall to the ground. Atonic seizures are often associated with Lennox-Gastaut syndrome, but are also associated with tuberous sclerosis complex, Dravet syndrome, Doose syndrome, Aicardi syndrome, CDKL5 deficiency, and Dupl5q syndrome.

In some embodiments, animal seizure models can be used to determine treating or mitigating a seizure. In some embodiments, the animal seizure model can be, but is not limited to, a model of generalized seizures, a model of limbic seizures, a distinct seizure model in an animal rendered epileptic by kindling, a model of ongoing seizures, and a model wherein the animal is subject to electrical shock to include tonic convulsions. In some embodiments, the animal seizure model can be, but is not limited to, a maximal PTZ seizure model, a 6 Hz model, a corneal kindled mouse model, a pilocarpine induced status epilepticus model, and a maximal electroshock model.

### B. Method of Reducing Frequency of Seizures

In some embodiments, the present invention provides a method of reducing epilepsy. In some embodiments, the present invention provides a method of reducing the frequency of seizures.

In some embodiments, animal studies can be used to determine reducing the frequency of seizures. In some embodiments, animal studies include but are not limited to, rodents, rats, mice, zebrafish. In some embodiments, the animal study comprises administering a compound of the present invention about 1 hour, about 2 hours, about 3 hours, about 4 hours, or about 5 hours before administering the convulsant drug. In some embodiments, the animal study comprises administering a compound of the present invention about 1 hour before administering the convulsant drug.

In some embodiments, animal seizure models can be used to determine reducing the frequency of seizures. In some embodiments, the animal seizure model can be, but is not limited to, a model of generalized seizures, a model of limbic seizures, a distinct seizure model in an animal rendered epileptic by kindling, a model of ongoing seizures, and a model wherein the animal is subject to electrical shock to include tonic convulsions. In some embodiments, the animal seizure model can be, but is not limited to, a maximal PTZ seizure model, a 6 Hz model, a corneal kindled mouse model, a pilocarpine induced status epilepticus model, and a maximal electroshock model.

In some embodiments, the present invention provides a method of reducing the frequency of seizures, comprising administering to a subject in need thereof, a therapeutically effective amount of a compound of the present invention, or a pharmaceutically acceptable salt thereof, without inducing hypnotic effects in the subject, thereby reducing the frequency of seizures.

### C. Combination Therapy

The methods of the present invention can also include a second therapeutic agent for treating or mitigating a seizure or epilepsy. Combination therapy may involve administering the rapidly infusing composition formulated with CBG or derivative/analog thereof and a second therapeutic agent, such as one or more antiepileptic drugs (AEDs) (other than CBG or its derivatives/analogs) for the treatment of epilepsy. In particular, when co-administered with one or more AEDs, the compositions formulated with CBG or a derivative/analog thereof may advantageously function to reduce traditional AED usage, for example by reducing the number of AEDs dosed, reducing the overall dosage of the AED(s) needed, or shortening the AED dosage period, without sacrificing antiepileptic efficacy.

AEDs suitable for use in combination therapy may function as voltage-gated ion channel blockers, ligand-gated ion channel blockers, antagonists of the excitatory receptors for glutamate and N-methyl-D-aspartate, or enhancers of the γ-aminobutyric acid, and may be categorized as narrow-spectrum (typically used to treat focal seizures) or broad-spectrum (treats a variety of seizure varieties). Examples of AEDs include, but are not limited to, phenobarbital, eslicarbazepine, ethosuximide, everolimus, tiagabine, acetazolamide, brivaracetam, cenobamate, clobazam, clorazepate, lorazepam, methsuximide, primidone, diazepam, divalproex, felbamate, fenfluramine, carbamazepine, oxcarbazepine, lacosamide, vigabatrin, gabapentin, lamotrigine, pregabalin, baclofen, phenytoin, valproic acid or its salts such as sodium valproate, topiramate, zonisamide, levetiracetam, clonazepam, rufinamide, stiripentol, perampanel, and fosphenytoin.

Combination therapy is intended to embrace administration of these therapies in a sequential manner, that is, wherein the rapidly infusing composition and one or more other therapies are administered at a different time, as well as administration of these therapies, or at least two of the therapies, in a substantially simultaneous manner. Substantially simultaneous administration can be accomplished, for example, by administering to the subject multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, the rapidly infusing composition formulated with CBG or derivative/analog thereof may be administered via buccal administration while a second therapeutic agent of the combination may be administered intravenously. Alternatively, for example, all therapeutic agents may be administered buccally.

Combination therapy also can embrace the administration of the rapidly infusing composition in further combination with other biologically active ingredients and non-drug therapies. Examples of non-drug therapies may include, but are not limited to, surgery to remove a localized resectable section of brain tissue identified to cause seizures such as anterior temporal lobe resection to remove the hippocampus, removal of tumors, removing parts of the neocortex, and corpus callosotomy; and neurostimulation such as vagus nerve stimulation, anterior thalamic stimulation (deep brain stimulation), and closed-loop responsive stimulation. Where the combination therapy further comprises a non-drug treatment, the non-drug treatment may be conducted at any suitable time so long as a beneficial effect from the co-action of the combination of the therapeutic agent(s) and non-drug treatment is achieved. For example, in appropriate cases, the beneficial effect is still achieved when the non-drug treatment is temporally removed from the administration of the therapeutic agents, perhaps by days or even weeks.

### IV. Formulation and Administration

The compositions of the present invention can be prepared in a wide variety of oral, parenteral, thin film, and topical dosage forms. Oral preparations include tablets, pills, powder, dragees, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). The composition of this invention can also be administered by thin film drug delivery methods.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

The compounds of the present invention can also be in the salt forms, such as acid or base salts of the compounds of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the compounds of the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the compounds of the present invention mixed with a filler or binders such as lactose or starches, lubricants such as talc or magnesium stearate, and, optionally, stabilizers. In soft capsules, the compounds of the present invention may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compounds of the present invention are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compounds of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compounds of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compound of the present invention can be present in any suitable amount, and can depend on various factors including, but not limited to, weight and age of the subject, state of the disease, etc. Suitable dosage ranges for the compound of the present invention include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The compounds of the present invention can be administered at any suitable frequency, interval and duration. For example, the compound of the present invention can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful, or with adjunctive agents that may not be effective alone, but may contribute to the efficacy of the active agent.

The compounds of the present invention can be co-administered with another active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, i.e., preparing a single pharmaceutical composition including both the compound of the present invention and the active agent. In other embodiments, the compound of the present invention and the active agent can be formulated separately.

The compound of the present invention and the active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the other active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present invention and the active agent are suitable in the compositions and methods of the present invention.

### V. Examples

### Example 1

Animals. Male NIH Swiss or CF1 mice (Charles-Rivers, 22-34g) were used in the present study. In most of the experiments, male CF1 mice were used. In the experiment exploring the strain difference between CBD and CBG efficacy in the MES test, these compounds were also tested in male NIH Swiss mice. All experiments involving animals complied with the ARRIVE guidelines of the National Centre for the Replacement, Refinement, and Reduction of Animals in Research and were performed in accordance with the National Institutes of Health Guide for the Care and Use of Laboratory Animals (NIH publication No. 8023, revised in 1978) under protocols approved by the University of California, Davis, Institutional Animal Care and Use Committee.

All the cannabinoids were prepared in ethanol: Cremophor: saline in the ratio of 2:1:17. These were administered Intraperitoneally (IP) in a dose volume of 10 ml/kg.

Mouse maximal electroshock test. Samples of CBD, both from decarboxylation of cannabidiolic acid (CBDA) and a commercial source, were tested in parallel as a comparator. Samples of CBD and CBG were prepared by decarboxylating their respective active precursors CBDA and cannabigerolic acid (CBGA; 95+% pure) by heating at 102° C. (215° F.) for 60 min. The precursor compounds were isolated from hemp. For comparison, the antiseizure activity of pure, commercial, hemp-derived CBD and chemically synthesized CBG in crystalline form was also determined in the MES test. Also investigated was whether CBDA and CBGA have antiseizure potential at the highest dose tested (150 mg/kg) in this model. Male CF1 mice were used in the current study. Sixty min after IP injection of the cannabinoids, the animals were subjected to a 0.2-s, 60-Hz, 50-mA electrical stimulus delivered with corneal electrodes (5 mm diameter stainless steel balls) wetted with 0.9% saline. Animals failing to show THLE were scored as protected. In the time-response curve experiment, CBG was administered at different times before the MES test. In most of the experiments, male CF1 mice were used. In the experiment exploring the strain difference between CBD and CBG efficacy in the MES test, these compounds were additionally tested in male NIH Swiss mice.

CBD obtained from CBDA isolates protected animals from THLE at doses between 25-150 mg/kg with an ED50 value of 62.7±5.6 mg/kg. CBG obtained from CBGA was also protective in a similar range of doses 65-200 mg/kg with an ED50 value of 99.03±7.01 mg/kg. A commercial sample of crystalline, hemp-derived CBD and crystalline, synthetic CBG had similar efficacies and potencies. The ED50 value of commercial CBD was 83.7±5.7 mg/kg. Pure crystalline CBG protected 62.5%, 75% and 62.5% of animals at 100 mg/kg, 150 mg/kg and 200 mg/kg. In contrast, CBC oil was less potent than CBD and CBG. At doses of 50, 100, 150 and 250 mg/kg, the fraction of animals protected was 11.1%, 12.5%, 12.5% and 25%, respectively. CBDA had no antiseizure activity at the highest dose tested whereas CBGA protected 25% of animals tested.

TABLE 1
Effect of different cannabinoids (CBDA, CBGA, pure crystalline
chemically synthesized CBG, crystalline hemp-derived CBD,
and CBC oil) in the MES test using male CF1 mice. Cannabinoids
were administered 60 min before the challenge. Each group
represents eight mice. Both pure crystalline chemically
synthesized CBG and crystalline hemp-derived CBD were
effective in the mouse MES test.

| | Dose | Percentage | ED50 |
|---|---|---|---|
| Cannabinoids | (mg/kg) | Protection | (mg/kg, IP) |
| Cannabidiolic acid (CBDA) | 150 | 0 | |
| Cannabigerolic acid (CBGA) | 150 | 25 | |
| Pure crystalline chemically | 100 | 62.5 | |
| synthesised cannabigerol | 150 | 75 | |
| (CBG) | 200 | 62.5 | |
| Crystalline hemp derived | 50 | 0 | 83.7 ± 5.7 |
| CBD | 65 | 37.5 | |
| | 75 | 37.5 | |
| | 85 | 50 | |
| | 100 | 75 | |
| | 150 | 75 | |
| CBC oil | 50 | 11.1 | |
| | 100 | 12.5 | |
| | 150 | 12.5 | |
| | 200 | 25 | |

TABLE 2
Strain differences in the antiseizure effect of CBG and CBD
in mouse electroshock test (0.2-s, 60-Hz, 50-mA). Treatments
were administered 1 h before maximal electroshock challenge.

| | | | Percentage protection from | |
|---|---|---|---|---|
| Treatment | Strain | Dose | Tonic Hind limb Extension | |
| CBD | Male NIH | 100 | 12.5 | (1/8) |
| | Swiss mice | 150 | 12.5 | (1/8) |
| | Male CF1 mice | 100 | 100 | (8/8) |
| | | 150 | 100 | (8/8) |
| CBG | Male NIH | 100 | 12.5 | (1/8) |
| | Swiss mice | 150 | 12.5 | (1/8) |
| | Male CF1 mice | 100 | 62.5 | (5/8) |
| | | 150 | 75 | (6/8) |

Both CBD and CBG were effective in male CF1 mice strain, and not protective in male NIH Swiss mice.

Mouse pentylenetetrazol (PTZ) test. Sixty min after IP injection of either cannabidiol (CBD) or cannabigerol (CBG) in male CF1 mice, the animals were challenged with 80 or 100 mg/kg PTZ delivered via intraperitoneal (IP) route of administration. The onset of myoclonic jerks, generalized clonus, tonus/tonic extension, and mortality was observed. The percentage of animals was further calculated that underwent these phases caused by PTZ. Failure to observe such phases or delayed onset is considered as protection.

TABLE 3
Effect of CBD or CBG on PTZ (IP)-induced seizures in male CF1 mice. Treatments were administered 1 h before the PTZ
challenge. The onset and percentage of animals undergoing different phases of PTZ (jerks, generalized clonus, tonus,
and death) seizures were measured. The numbers in brackets represent the number of animals that showed seizure phase
out of the total number of animals tested. Animals were observed 1 h post PTZ for these seizure signs.

| | | Percentage | | Percentage | | | | |
|---|---|---|---|---|---|---|---|---|
| | | of animals | | animals | | Percentage | | |
| | | having | Onset of | having | | animals | | |
| Treatment | Onset of | myoclonic | generalized | generalized | Onset of | having | Death | Percentage |
| (mg/kg) | jerks (s) | jerks | clonus (s) | clonus | tonus (s) | tonus | time (s) | mortality |

PTZ (80 mg/kg, IP)

| Vehicle | 71.5 ± 4.3 | 100 | (8/8) | 127.6 ± 18.6 | 100 | (8/8) | None | None | | None | 0 |
|---|---|---|---|---|---|---|---|---|---|---|---|
| CBD (100 | 138.1 ± 61.9 | 100 | (8/8) | 333.9 ± 174.3 | 87.5 | (7/8) | None | None | | None | 0 |
| mg/kg, IP) | | | | | | | | | | | |
| CBD (150 | 95.6 ± 13.3 | 100 | (8/8) | 612.5 ± 515.5 | 25 | (2/8)^{a} | None | None | | None | 0 |
| mg/kg, IP) | | | | | | | | | | | |
| CBG (100 | 101.1 ± 50.7 | 87.5 | (7/8) | 199.1 ± 90.6 | 87.5 | (7/8) | None | None | | None | 0 |
| mg/kg, IP) | | | | | | | | | | | |
| CBG (150 | 93 ± 26.6 | 100 | (8/8) | 117.8 ± 46.8 | 62.5 | (5/8)^{b} | 852.5 ± 170.5 | 25 | (2/8) | None | 0 |
| mg/kg, IP) | | | | | | | | | | | |

PTZ (100 mg/kg, IP)

| | | | | | | | | | | | | |
|---|---|---|---|---|---|---|---|---|---|---|---|---|
| Vehicle | 43.4 ± 2.5 | 100 | (8/8) | 56.8 ± 4.8 | 100 | (8/8) | 467.4 ± 115.3 | 87.5 | (7/8) | 1454.9 ± 248.9 | 87.5 | (7/8) |
| CBD (150 | 63.6 ± 4.8** | 100 | (8/8) | 329.3 ± 152.3 | 100 | (8/8) | 1362.5 ± 888.5 | 25 | (2/8) | None | 0 | (0/8) |
| mg/kg, IP) | | | | | | | | | | | | |
| CBG (150 | 50.1 ± 3.3 | 100 | (8/8) | 200.8 ± 105.9 | 100 | (8/8) | 1351.3 ± 410.7 | 100 | (8/8) | 1082.6 ± 265.5 | 62.5 | (5/8) |
| mg/kg, IP) | | | | | | | | | | | | |

^{a}One additional animal, besides two animals displaying generalized clonus, at this dose showed tremors and jerks starting at 1 min 19 sec after PTZ, but there was no clear-cut generalized clonus phase.
^{b}Three additional animals, besides five animals displaying generalized clonus, in this dose did not have clear-cut clonus. Instead, they were seizing intermittently and continuous, characterized like status epilepticus. The symptoms included tail wiggling, head bobbing, jerking, simultaneously fall on their back, and having tremors.

Conclusion from PTZ (IP test). CBD (150 mg/kg, IP) was found to be effective in protecting animals from generalized clonus in low dose PTZ test (80 mg/kg, IP). CBD (150 mg/kg, IP) increased the latency to onset of myoclonic jerks and protected animals from undergoing tonus phase and mortality in high dose PTZ test (100 mg/kg). There was no difference in terms of generalized clonus by CBD at high dose PTZ test. CBG in contrast to CBD had no effect in PTZ seizure test, except at high dose it protected some animals from mortality.

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating or mitigating a seizure or epilepsy, comprising administering to a subject in need thereof, a therapeutically effective amount of a compound of Formula I: 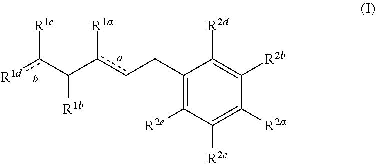
or a pharmaceutically acceptable salt thereof, thereby treating the seizure or epilepsy, wherein:
R^{1a} and R^{1d} are each independently —CO₂R^{1e}, C₁₋₂₀ alkyl, C₂₋₂₀ alkenyl or C₂₋₂₀ alkynyl, wherein at least one of R^{1a} or R^{1d} is methyl or isopropyl;
R^{1b} and R^{1c} are each independently hydrogen or oxygen;
alternatively, when R^{1b} is oxygen, R^{1b} is combined with R^{1a} and the atoms to which they are attached to form an epoxide ring;
alternatively, R^{1b} is combined with R^{1d} and the atoms to which they are attached to form a C₄₋₈ cycloalkyl, wherein the cycloalkyl is substituted with 1-3 R^{1e} groups;
R^{1e} is H, C₁₋₂₀ alkyl, C₂₋₂₀ alkenyl or C₂₋₂₀ alkynyl;
R^{2a} is —OR^{2F}, C₁₋₂₀ alkyl, C₂₋₂₀ alkenyl or C₂₋₂₀ alkynyl;
R^{2b} and R^{2e} are each independently hydrogen, halogen, —OR^{2f}, —NR^{2f}R^{2g}, or —C(O)OR^{2f};
R^{2d} and R^{2e} are each independently —OH, —OC(O)R^{2f}, —OR^{2F}, C₁₋₂₀ alkyl, C₂₋₂₀ alkenyl or C₂₋₂₀ alkynyl;
R^{2f} and R^{2g} are each independently hydrogen, C₁₋₂₀ alkyl, C₂₋₂₀ alkenyl or C₂₋₂₀ alkynyl; and
dashed lines a and b are each independently absent or a bond.

2. The method of claim 1, wherein:
R^{1a} and R^{1d} are each independently —CO₂R^{1e}, C₁₋₂₀ alkyl, C₂₋₂₀ alkenyl or C₂₋₂₀ alkynyl, wherein at least one of R^{1a} or R^{1d} is methyl or isopropyl;
R^{1b} and R^{1c} are each independently hydrogen or oxygen;
alternatively, when R^{1b} is oxygen, R^{1b} is combined with R^{1a} and the atoms to which they are attached to form an epoxide ring;
alternatively, R^{1b} is combined with R^{1d} and the atoms to which they are attached to form a C₄₋₈ cycloalkyl, wherein the cycloalkyl is substituted with 1-3 R^{1e} groups;
R^{1e} is H, C₁₋₂₀ alkyl, C₂₋₂₀ alkenyl or C₂₋₂₀ alkynyl;
R^{2a} is —OR^{2f}, C₁₋₂₀ alkyl, C₂₋₂₀ alkenyl or C₂₋₂₀ alkynyl;
R^{2b} and R^{2c} are each independently hydrogen, halogen, —OR^{2f}, or —NR^{2f}R^{2g};
R^{2d} and R^{2e} are each independently —OH, —OC(O)R^{2f}, —OR^{2f}, C₁₋₂₀ alkyl, C₂₋₂₀ alkenyl or C₂₋₂₀ alkynyl;
R^{2f} and R^{2g} are each independently hydrogen, C₁₋₂₀ alkyl, C₂₋₂₀ alkenyl or C₂₋₂₀ alkynyl; and
dashed lines a and b are each independently absent or a bond.

3. The method of claim 1 or 2, wherein R^{1a} and R^{1d} are each independently C₁₋₂₀ alkyl.

4. The method of claim 1 or 2, wherein Ria is C₁₋₂₀ alkyl, and R^{1d} is C₂₋₂₀ alkenyl.

5. The method of claim 1 or 2, wherein R^{1d} is C₁₋₂₀ alkyl or C₂₋₂₀ alkenyl.

6. The method of any one of claims 1 to 6, wherein R^{1b} and R^{1c} are each hydrogen.

7. The method of any one of claims 1 to 6, wherein R^{2a} is C₁₋₂₀ alkyl.

8. The method of any one of claims 1 to 7, wherein R^{2b} and R^{2c} are each hydrogen.

9. The method of any one of claims 1 to 7, wherein
R^{2d} and R^{2e} are each independently —OH, —OC(O)R^{2f}, or —OR^{2f}; and
R^{2f} is hydrogen, or C₁₋₂₀ alkyl.

10. The method of any one of claims 1 to 9, wherein R^{2d} and R^{2e} are each —OH.

11. The method of claim 1 or 2, wherein:
R^{1a} and R^{1d} are each independently C₁₋₂₀ alkyl, wherein at least one of R^{1a} or R^{1d} is methyl or isopropyl;
R^{1b} and R^{1c} are each hydrogen;
R^{2a} is —OR²¹ or C₁₋₂₀ alkyl;
R^{2b} and R^{2c} are each independently hydrogen, or —C(O)OR^{2f};
R^{2d} and R^{2e} are each independently —OH, —OC(O)R^{2f}, or —OR^{2f};
each R^{2f} and R^{2g} is hydrogen, or C₁₋₂₀ alkyl; and
dashed lines a and b are each independently absent or a bond.

12. The method of claim 1 or 2, wherein:
R^{1a} and R^{1d} are each independently C₁₋₂₀ alkyl, wherein at least one of R^{1a} or R^{1d} is methyl or isopropyl;
R^{1b} and R^{1c} are each hydrogen;
R^{2a} is —OR²¹ or C₁₋₂₀ alkyl;
R^{2b} and R^{2c} are each independently hydrogen, or —C(O)OH;
R^{2d} and R^{2e} are each —OH;
R^{2f} is C₁₋₂₀ alkyl; and
dashed lines a and b are each independently absent or a bond.

13. The method of claim 1 or 2, where the compound is Formula Ia: 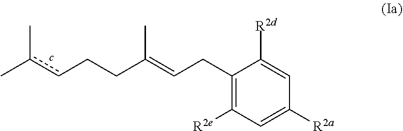
or a pharmaceutically acceptable salt thereof,
wherein
R^{2a} is C₁₋₂₀ alkyl, C₂₋₂₀ alkenyl or C₂₋₂₀ alkynyl;
R^{2d} and R^{2e} are each independently —OH, —OC(O)R^{2f}, or —OR^{2f};
each R^{2f} is independently hydrogen, C₁₋₂₀ alkyl, C₂₋₂₀ alkenyl or C₂₋₂₀ alkynyl; and
dashed line c is absent or a bond.

14. The method of any one of claims 1 to 13, wherein the compound is Formula Ib: 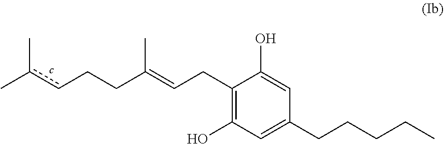
or a pharmaceutically acceptable salt thereof,
wherein dashed line c is absent or a bond.

15. The method of any one of claims 1 to 13, wherein the compound is: 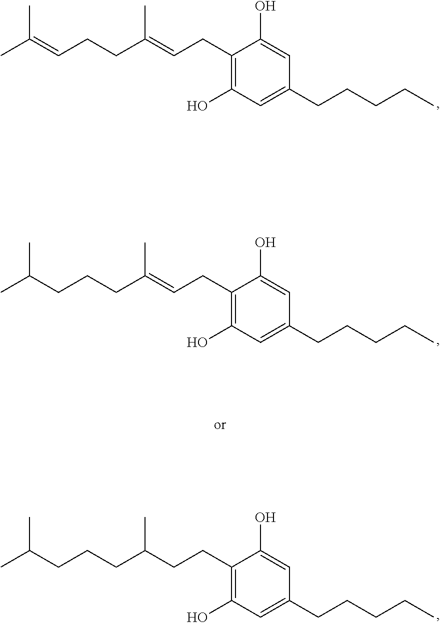
or a pharmaceutically acceptable salt thereof.

16. The method of any one of claims 1 to 15, wherein the compound is: 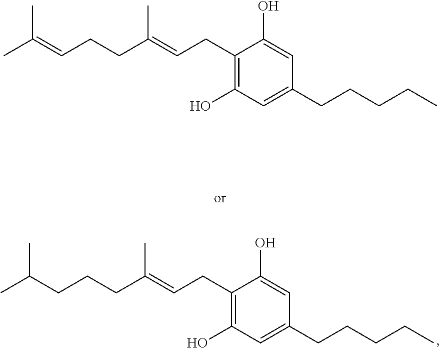
or a pharmaceutically acceptable salt thereof.

17. The method of any one of claims 1 to 16, wherein the compound of Formula I is cannabigerol: 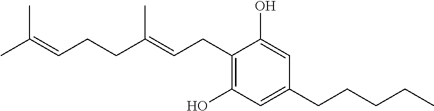 [SMILES: CCCCCc1cc(O)c(C/C=C(\C)CCC=C(C)C)c(O)c1]
or a pharmaceutically acceptable salt thereof.

18. The method of any one of claims 1 to 17, wherein the compound of Formula I is cannabigerol:

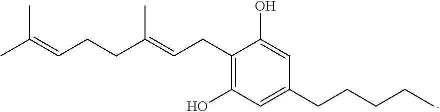 [SMILES: CCCCCc1cc(O)c(C/C=C(\C)CCC=C(C)C)c(O)c1]

19. A method of reducing the frequency of seizures, comprising administering to a subject in need thereof, a therapeutically effective amount of a compound of any one of claims 1 to 18, or a pharmaceutically acceptable salt thereof, without inducing hypnotic effects in the subject, thereby reducing the frequency of seizures.
